(19) United States
(12) Patent Application Publication
(10) Pub. No.: US 2024/0027426 A1
(43) Pub. Date: Jan. 25, 2024

(54) Method for determining the alcohol content in exhaled air

(71) Applicant: ALKO PREVENT, WARSZAWA (PL)

(72) Inventor: Vasyl Rakivnenko, Warszawa (PL)

(21) Appl. No.: 17/873,023

(22) Filed: Jul. 25, 2022

(51) Int. Cl. G01N 33/497; G06N 3/04; G06F 21/32

(52) U.S. Cl. CPC G01N 33/4972; G06N 3/0454; G06F 21/32

(57) ABSTRACT

A method of remote control of the alcohol content in the exhaled air is described. The alcohol concentration in the exhaled air is measured by means of a control device with a radio data transmission ability. The created set reference data including identifiers of the device and the user is stored in a web application data collection module on a server contained inside the network data analysis infrastructure. It is then used to train the deep model a convolutional neural network whose task is to classify and identify the characteristic visual features of the individual faces of users, individual features identifying the control device. This information being compared with the value of the alcohol concentration in the exhaled air obtained from the control device with the date, time, geographical location to verify alcohol readings and to take actions such as preventing access to a workplace.

The subject of the invention is a method of remotely evaluating the alcohol content in the exhaled air, applicable for conditioning access to workplaces using a computer application. The creation of an appropriate model allows the development of a virtual assistant supporting the work of a person supervising sobriety at the workplace.

So far, methods of remote monitoring and verifying the sobriety of employees are known, including remote measurement of alcohol content in exhaled air and informing the employer about its result based on telecommunications systems, with the possibility of remotely blocking access to the workplace. Known solutions do not include technology that enables the detection of counterfeits, as well as mistakes in the identification of the person for whom the measurement is made, with the possibility of self-learning.

As part of the solution, according to the invention, a set of activities was developed, including data preparation, transformation, and analysis to create an optimal device and user identification model.

From the Canadian description of the invention CA2906116 there is known a method of monitoring sobriety by means of a handheld device for breath testing, which, after receiving the alcohol concentration value in the exhaled air, generates a signal containing data on the content of the breath.

The substance and user identification data are sent wirelessly via the cellular network to the receiving station.

The American invention US2020191769A1 discloses a method of remotely monitoring sobriety by means of wireless devices connected via a communication network, providing users with various forms of alerts in the form of electronic messages.

From another US description of the invention, US2020256848, a user sobriety monitoring system is known. The system may include a test device that generates a signal on the content of the substance. The test device may further include a mouthpiece and a user identification device. The user identification device may generate user identification data in response to the user's breath and may transmit it from the testing device to the monitoring station. The test device may further include at least one LCD screen or a light emitting diode (“LED”). At least one of the LCD screens or the LEDs may display at least one randomly generated visible identification mark.

On the other hand, from also the American description of the invention US2020388117 there is known a method of monitoring sobriety by means of a handheld breath testing device which, after receiving a user's breath, generates a signal containing the substance content data and user identification data and wirelessly transmits the signal to the receiving station, in which the breath testing device includes a fingerprint reader.

### BRIEF DESCRIPTION OF THE DRAWING

FIG. 1 depicts an overview of one embodiment of the invention.

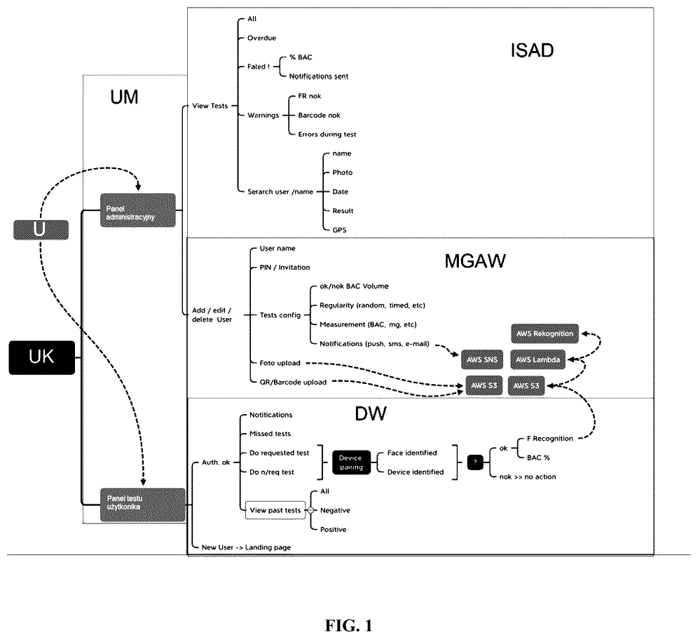

### DETAILED DESCRIPTION

The aim of the invention is to develop a method for measuring the alcohol content in the exhaled air of users, leveling the possibility of a false and incorrect measurement as to the device and user.

Solving the problem of automatic identification according to the proposed method allows for continuous evaluation of the recognition model.

As used herein, the word control refers to verification and taking into account a factor, in this case, the factor being the level of exhaled alcohol.

A method of remotely monitoring the alcohol content in the exhaled air, in which the alcohol concentration in the exhaled air is measured by means of a control device (UK) with radio data transmission technology, characterized in that the electronic traces of the control device (UK) and user (U) are saved in the web application data collection module (MGAW) on the server contained within the data analysis network infrastructure (ISAD), which are then used to train the deep convolutional neural network model, whose task is classification and identification of the characteristic visual features of users' faces (U), individual features identifying the control device (UK), such as dimensions and an individual QR code or bar code, and this information is compared with the alcohol concentration value obtained from the control device (UK) in exhaled air, combined in the mobile device (UM) with the date, time, geographical location, and with said defined QR code or barcode individualizing the electronic trace of the control device (UK) and visual characteristics of the user obtained from the camera of said mobile device (UM) (U), additionally with fingerprints, an individual code personalizing the user (U) manually entered into a mobile device (UM), then the obtained information is digitized and, via telecommunications links, saved in any format on the above-mentioned server contained within the data analysis network infrastructure (ISAD) in the web application data collection module (MGAW), for the purpose of comparison with model information, whereby via telecommunications links from said server, push or text messages are sent to the mobile device (UM) about the need to make a test, at any time intervals, preferably at the same time of the day and time, obliging the user (U) to perform measurement activities, provided that access to the workplace is allowed or prevented, and if the measured result of the alcohol concentration measurement in the exhaled air (U) by the user (U) is within the tolerance limits set in (BGAW), then the data analysis network infrastructure (ISAD) server sends the information unlocking the access to the workplace,
if the measured result of the measurement of alcohol concentration in the exhaled air (U) by the user (U) is above in the data collection block of the web application (BGAW) set with tolerance ranges, then from the data analysis network infrastructure (ISAD) server information is sent blocking access to a workplace,
if the data analysis network infrastructure (ISAD) server does not receive from the control device (UK) via the mobile device (UM) within the set period, the feedback on the user's breath alcohol concentration (UK) or receives incomplete information, including only the concentration measurement result exhaled alcohol with an incomplete individualizing electronic trace of the control device (UK) and the user (U), then the server sends information blocking access to the workplace,
if the measured control device (UK) result of the measurement of the alcohol concentration in the exhaled air (U) by the user complies or exceeds the tolerance set in the data collection block of the web application (BGAW), and the electronic trace of the control device identifying the device or the user is inconsistent, then from the server information that blocks access to the workplace is sent.

Preferably, in the web application data collection module (BGAW), individual user accounts (U) are created in which historical data, including date, individual device trace (UK) and user (U), and measured blood alcohol concentration results are stored.

Preferably, the web application data collector (BGAW) establishes a measurement time interval, including time of day, date, time, or time between preset measurement times, or random measurement at any time period, with the breath alcohol tolerance limit.

Preferably, the workstation is a means of transport or an element of production infrastructure, in particular a car, a production machine, a warehouse cart, an airplane, a production machine, or entrance gates, the server is connected to an electronic ignition or access control device.

Preferably, the deep convolutional neural network model for user face recognition (U) as part of the data analysis network infrastructure (ISAD) is continuously trained, that is, when new data arrives in the data block
storing reference data (DW), in the next training iteration, the neural network model is trained using more examples.

Preferably, in the web application data collection module (BGAW), a model image of the user's face (U) is created on the basis of graphic files, presumably obtained from the camera of the mobile device (UM), and in time intervals it is compared with the image obtained within the individualizing electronic trace of the control device (UK) and the user (U), conditioning its compliance with the access to the workstation of the said user (U), the compliance being determined by the percentage of the characteristic visual elements of the user's face (U), which is stored in the template data storage module (DW) which can then be used to train a deep convolutional neural network model that determines the characteristics of the user's face (U) in percentage ranges.

Preferably, the email addresses or telephone numbers are added to the web application data collection module (BGAW), which will be notified if the breath alcohol concentration measurement result is above the tolerance set in the web application data collection module (BGAW), or if in the set over time, there is no feedback or the information obtained within the individualizing electronic trace of the control device (UK) and the user (U) is incomplete or inconsistent with the model information, in particular, the percentage of the characteristic visual elements of the user's face (U) is below the assumed value.

Preferably, the percentage of the visual characteristic of the user's face (U) is established within the framework of biometric 3D authentication of the personality of the user's image (U).

Preferably, if the user (U) ignores the command of the data analysis network infrastructure (ISAD) server regarding the measurement of alcohol concentration in the breath and positive or negative facial recognition, then along with the blocking of the workstation, said server sends an email notification or a text message to the addresses defined in the module web application data collection (BGAW).

The subject of the invention has been presented in the embodiment in the drawing, which shows a block diagram illustrating the method.

The use of the invention allows for a very accurate correlation of the user, the measuring device and the result of measuring the alcohol concentration in the exhaled air, with a very short time of at most a dozen or so seconds. The “learned” models are based on model data, then they are used to identify the features of individual faces and the features of individual devices. The selection of models is performed with the use of appropriate evaluation criteria and the selection of the best model obtained so far. The entire process of optimizing new models is automated and requires no human intervention.

A method of remotely checking the alcohol content in the exhaled air, in which the alcohol concentration in the exhaled air is measured by means of a control device (UK) with radio data transmission technology, characterized by the fact that the created reference data set (DW) containing the individualizing electronic traces of the control device (UK) and the user (U) are saved in the web application data collection module (MGAW) on the server contained within the data analysis network infrastructure (ISAD), which are then used to train the deep convolutional neural network model, which the task is to classify and identify the characteristic visual features of the users' faces (U), individual features identifying the control device (UK), such as dimensions and an individual QR code or bar code, and this information is compared with the concentration value obtained from the control device (UK) breath alcohol, combined in the mobile device (UM) with the date, time, geographic location, and with said defined QR code or barcode individualizing being the electronic trace of the control device (UK) and features obtained from the camera of said mobile device (UM) with the user's data (U), additionally with fingerprints, an individual code personalizing the user (U) manually entered into the mobile device (UM), then the obtained information is digitized and via telecommunications links are saved in any format on the above-mentioned server contained within the network infrastructure data analysis (ISAD) in the web application data collection module (MGAW), in order to compare them with the model information, whereby using telecommunications links from the said server, push or text messages are sent to the mobile device (UM) about the need to take a test, at any time intervals, preferably at the same time of day and time, obliging the user (U) to
perform measurement activities on the condition that access to the workplace is allowed or prevented, and
if the measured result of the alcohol concentration measurement in the exhaled air (U) by the user (U) is within the tolerance limits set in (BGAW), then the data analysis network infrastructure (ISAD) server sends the information unlocking the access to the workplace,
if the measured result of the measurement of alcohol concentration in the exhaled air (U) by the user (U) is above in the data collection block of the web application (BGAW) set with tolerance ranges, then from the data analysis network infrastructure (ISAD) server information is sent blocking access to the workplace,
if the data analysis network infrastructure (ISAD) server does not receive from the control device (UK) via the mobile device (UM) within the set period of time the feedback on the user's breath alcohol concentration (UK) or receives incomplete information including only the result of the alcohol concentration measurement in exhaled with an incomplete individualizing electronic trace of the control device (UK) and user (U), then the server sends information blocking access to the workplace,
if the measured test device (UK) result of the measurement of the alcohol concentration in the user's exhaled (U) air matches or exceeds the tolerance set in the web application data collection block (BGAW), and the electronic trace of the control device identifying the device is not compliant, or the user, the server sends information blocking access to the workplace, if the user (U) ignores the command of the data analysis network infrastructure (ISAD) server regarding the measurement of alcohol concentration in exhaled air and positive or negative facial recognition, then along with blocking the workstation, the said server sends an email notification or text message to the addresses defined in the data collection module web application (BGAW).

In the web application data collection module (BGAW), individual user accounts (U) are created in which historical data including date, individual device trace (UK) and user (U), the result of the measured blood alcohol concentration are saved. In the web application data collection module (BGAW), the time interval of the measurement is set, including the time of day, date, time, or the time between the preset measurement times, or the randomness of the measurement at any time, with the limit value of alcohol tolerance in the exhaled air. A workstation is a means of transport or an element of production infrastructure, in particular a car, production machine, warehouse cart, plane, production machine or entrance gates, where the server is connected to an electronic ignition or access control device. The deep convolutional neural network model for user face recognition (U), which is part of the data analysis network infrastructure (ISAD), is continuously trained, which means that if new data appears in the template data storage (DW) block, in the next iteration training, the neural network model is trained using more examples. In the web application data collection module (BGAW), a model face image is created on the basis of graphic files obtained by default from the camera of a mobile device (UM) user (U) and in time intervals it is compared with the image obtained as part of the individualizing electronic trace of the control device (UK) and the user (U), conditioning access to the workstation of said user (U), the compliance being determined by a percentage characteristic visual elements of the user's face (U), which is saved in the template data storage (DW) module, which can then be used to train a deep convolutional neural network model, whose task is to determine the characteristics of the user's face (U) in percentage ranges. To the web application data collection module (BGAW), email addresses or telephone numbers are added, which will be notified if the result of the breath alcohol concentration measurement is above the tolerance set in the web application data collection module (BGAW), or if it does not take place within a given period of time. has feedback or the information obtained within the individualizing electronic footprint of the control device (UK) and the user (U) is incomplete or inconsistent with the model information, in particular, the percentage of the characteristic visual elements of the user's face (U) is below the assumed value. The percentage of the characteristic visual elements of the user's face (U) is determined as part of the biometric 3D authentication of the personal features of the user's image (U).

### SUMMARY

The subject of the invention is a method of remote control of alcohol content in the exhaled air, applicable when conditioning access to workplaces using a computer application. Creating an appropriate model allows you to develop a virtual assistant supporting the work of a person supervising sobriety at the workplace. The method of remote control for the value of users' alcohol, in which the concentration of alcohol in the exhaled air is measured by means of a control device (UK) implicitly with radio data transmission technology, is characterized by the fact that the created data set the reference (DW) comprising the individualizing electronic traces of the control device (UK) and the user (U) is stored in the web application data collection module (MGAW) on a server contained inside the network data analysis (ISAD) rastructure, which are then used to train a model of a deep convolutional neural network, whose task is to classify and identify the characteristic visual features of the individual face of the user (U), individual features identifying the device control (UK) such as dimensions and an individual QR code or barcode, this information being compared with the value of the alcohol concentration in the exhaled air obtained from the control device (UK), combined in the device mobile (UM) with date, time, geographical location, and with the said
defined QR code or barcode individualizing the electronic trace of the control device (UK) and the user's visual features (U) obtained from the camera of the said mobile device (UM), in addition to fingerprints, individual user personalization code (U) entered manually into a mobile device (UM), then the obtained infograms are digitized and via telecommunications links are saved in any format on the server indicated above contained within the network data analysis infrastructure (ISAD) in the web application data collection module (MGAW), in order to compare them with the reference information, whereby push or SMS notifications are sent to the mobile device (UM) via telecommunications links from the said server to request the test, at any time intervals, preferably at the same time of day and time, obliging the user (U) to perform measurement activities provided that access to the workplace is allowed or prevented, whereby
if and measured with a control device (UK), the result of measuring the concentration of alcohol in the exhaled by the user (U) air is within the tolerance ranges determined in (MGAW), the server of the network data analysis infrastructure (ISAD) sends information unblocking access to the workplace,
if the result measured by the control device (UK) of the measurement of alcohol concentration in the exhaled by the user (U) air is above in the web application data collection block (MGAW) determined by tolerance intervals then from the server of the network data analysis infrastructure (ISAD) information blocking access to the workplace is sent,
if the Network Data Analysis Infrastructure (ISAD) server does not receive feedback on the user's exhaled alcohol (UK) or concentration from the control device (UK) via a mobile device (UM) within a set period of time, incomplete information, including only the result of the measurement of alcohol concentration in the exhaled with an incomplete individualizing electronic trace of the control device (UK) and the user (U), information is sent from the server blocking access to the workplace,
if the measurement of alcohol concentration in the exhaled air
(U) measured by the control device (UK) is consistent with or above the tolerance set in the web application data collection block (MGAW), and the trace is not compatible electronic control device identifying the device or user, information blocking access to the workplace is sent from the server.

1. A method of remote evaluation of the alcohol content in the exhaled air, in which the alcohol concentration in the exhaled air is measured by means of a control device (UK) by default with radio data transmission technology, characterized by the fact that the created set reference data (DW) including the individualizing electronic traces of the control device (UK) and user (U) is stored in the web application data collection module (MGAW) on a server contained inside the network data analysis infrastructure (ISAD), which is then used to train the deep model a convolutional neural network whose task is to classify and identify the characteristic visual features of the individual faces of users (U), individual features identifying the control device (UK) such as dimensions and individual QR code or a bar code, this information being compared
with the value of the alcohol concentration in the exhaled air obtained from the control device (UK), combined in the mobile device (UM) with the date, time, geographical location, and with the said defined QR code or barcode individualizing the electronic trace of the control device (UK) and the features obtained from the camera of the said mobile device (UM) visual user (U), in addition to fingerprints, an individual user personalization code (U) entered manually into a mobile device (UM), then the obtained information is digitized and saved via telecommunications links in any format on the server indicated above contained within the network data analysis infrastructure (ISAD) in the web application data collection module (MGAW), in order to compare it with the reference information, using telecommunications links from the said the server sends to the mobile device go (UM) push notifications or SMS about the need to do the test, at any time intervals, preferably at the same time of day and time, obliging the user (U) to perform the action measuring provided that access to the workplace is allowed or prevented, whereby
if the result of the measurement of alcohol concentration in the exhaled by the user (U) measured by the control device (UK) is within the tolerance ranges determined in (MGAW), information is sent from the server of the network data analysis infrastructure (ISAD) to unlock access to workstations,
if the result measured by the control device (UK) of the measurement of the concentration of alcohol in the exhaled by the user (U) air is in the above in the web application data collection block (MGAW) established tolerance intervals are used from the server of the network data analysis infrastructure (ISAD) to send information blocking access to the workplace,
if the network data analysis infrastructure (ISAD) server does not receive from the control device (UK) for via a mobile device (UM) in a fixed timed feedback period on the alcohol concentration in the exhaled air of the user (UK) or receives incomplete information including only the result of the measurement of alcohol concentration in the exhaled from incomplete individualizing electronic trace of the control device (UK) and the user (U), information blocking access to the workplace is sent from the server,
if the measurement of alcohol concentration in the exhaled air (U) measured by the control device (UK) is consistent with or above the tolerance set in the web application data collection block (MGAW) and the electronic trace of the device is not compatible the control person identifying the device or user is sent from the server to block access to the workplace.

2. The way to remotely evaluate the alcohol content of users, according to claim 1 is characterized by the fact that in the web application data collection module (MGAW) individual user accounts (U) are created in which historical data including date, individual device trace (UK) and user (U), the result of the concentration of measured alcohol in the blood.

3. The way to remotely evaluate the alcohol content of users, according to claim 1 is characterized by the fact that the web application data collection module (MGAW) sets the measurement time interval, including the time of day, date, time, or time between the set measurement hours, or the randomness of the measurement in any time period, with a limit of alcohol tolerance in the exhaled air.

4. The way to remotely evaluate the alcohol content of users, according to claim 1 notable by the fact that a workplace is a means of transport or an element of production infrastructure, in particular a car, a production machine, a warehouse trolley, an airplane, a production machine or entrance gates, whereby the server is connected to an electronic ignition or access control device.

5. The method of remote evaluate of the alcohol content of users, according to claim 1 is significant in that the model of a deep convolutional neural network for recognizing the user's face (U), which is part of the network analysis infrastructure data (ISAD) is trained continuously, which means that in the event that new data appears in the master data storage block (DW), in the next iteration of training, the neural network model is trained using more examples.

6. The way to remotely evaluate the alcohol content of users, according to claim 1 is characterized by the fact that in the web application data collection module (MGAW) a model image is created on the basis of graphic files implicitly obtained from the camera of a mobile device (UM). the face of the user (U) and at time intervals it is compared with the image obtained as part of the individualizing trace of the electronic control device (UK) and the user (U), conditioning its compliance access to the workplace of the said user (U), this compliance being determined by the percentage of characteristic visual elements of the user's face (U), which is saved in the module storing the reference data (CC), which can then be used to train the deep model
a convolutional neural network, whose task is to determine in percentage ranges the characteristics of the user's face (U).

7. The way to remotely evaluate the alcohol content of users, according to claim 1 is notable by the fact that the email addresses or telephone numbers to be sent to the web application data collection module (MGAW) are added if the result of the measurement of the alcohol concentration in the exhaled air is above the tolerance set in the web application data collection module (MGAW), or if there is no feedback within the given period or the information obtained within the individualizing trace of the electronic control device (UK) and the user (U) is incomplete or inconsistent with the reference information, in particular determined by the participation the percentage of characteristic visual elements of the user's face (U) is below the assumed value.

8. The method of remote evaluate of the content of the users' alcohol, according to claim 5, is characterized by the fact that the percentage of characteristic visual elements of the user's face (U) is determined within the framework of biometric 3D authentication of the individual features of the image user (U).

9. The method of remote evaluate of the content of the users' alcohol, according to claim 6, is characterized by the fact that the percentage of characteristic visual elements of the user's face (U) is determined within the framework of biometric 3D authentication of the individual features of the image user (U).

10. The method of remotely evaluate the alcohol content of users, according to claim 1, is characterized by the fact that if the user (U) ignores the command of the server of the network infrastructure for data analysis (ISAD) to perform a measurement of the alcohol concentration in the exhaled air and positive or negative facial recognition, along with blocking the workplace, the said server sends an email notification or a text message to the addresses defined in the web application data collection module (MGAW).
